(19) United States
(12) Patent Application Publication
(10) Pub. No.: US 2021/0077029 A1
(43) Pub. Date: Mar. 18, 2021

(54) Patient Monitoring System and Apparatus

(71) Applicant: Essential Healthcare Solutions (UK) Ltd, Birstall Yorkshire (GB)

(72) Inventor: Thomas Owens, Birstall Yorkshire (GB)

(21) Appl. No.: 16/959,469

(22) PCT Filed: Jan. 4, 2019
(86) PCT No.: PCT/GB2019/050023

§ 371 Date: Jul. 1, 2020

(30) Foreign Application Priority Data
Jan. 4, 2018 (GB) 1800120.6

(51) Int. Cl. A61B 5/00; A61B 5/0205; A61B 5/11; G16H 10/60

(52) U.S. Cl. CPC A61B 5/6892; A61B 5/02055; G16H 10/60; A61B 5/742; A61B 5/1116

(57) ABSTRACT

Apparatus includes a mat having a surface which contacts with a support means and opposing surface located with respect to a part of a person so that at least one parameter of the person is monitored by sensor means with the mat. The sensor means are connected with processing means to allow data relating to the at least one parameter to be collected and processed to generate a value which is indicative of the parameter at the time of the data collection and hence an indication of the condition of the person. The mat includes a plurality of spaced apart sensor means locations to provide an accurate collection of data for different parts and zones relating to the patient on a real time basis and make this available to care givers for the person.

The invention to which this application relates is a system and method for the monitoring of the condition of a person, and most typically a patient in a health care environment, so as to allow at least one parameter of the condition of the patient to be monitored in an effective manner and without, or allowing a reduction in the need for direct intervention by a member of staff caring for the patient.

It is known that, in practice, the monitoring of various parameters of the condition of a patient, and for example a plurality of patients in a hospital ward, is time consuming and labour intensive. It is also typically the case that on many occasions when the condition of the patient is checked, no health concerns are identified, but it is still necessary to monitor the patient's condition at regular intervals as a matter of course, as part of a recommended monitoring schedule and as part of good practice. Thus, conventionally, in order to achieve this, several staff members are employed and required to follow a particular monitoring schedule. The employment of the staff is expensive, can be problematic in being able to obtain a suitable number of qualified staff, and problematic in ensuring that all the staff are available as and when required and throughout each 24 hour period and all perform the monitoring tasks to an acceptable standard in order to be able to provide an accurate indication of the patient's condition.

Despite these problems and costs, it is generally accepted that these requirements have to be dealt with in order for normal and acceptable nursing standards to be achieved and to provide an acceptable level of care for the patient or groups of patients.

Another problem which exists in the field of patient care is being able to provide data which is “live” in that the data which is obtained in relation to the at least one parameter of the patient's condition provides an accurate indication of the condition at that instant of time and not at some historical time and in which the case the data is of little use. Furthermore there is often a need for eth data which is collected and which indicates the real condition of the patients to be available for subsequent reference, perhaps to show that an appropriate level of care had been provided, and/or to be used in conjunction with data relating to the care of other patients so that the data, in combination, can be used to provide analysis opportunities and the ability to provide improved care or care strategies going forward.

An aim of the present invention is therefore to provide apparatus and a method by which effective monitoring of the condition of a patient can be achieved whilst, at the same time reducing the requirement for direct human intervention to be involved in achieving an acceptable monitoring method and/or level of care.

A further aim of the invention is to provide data which is representative of a condition of at least one parameter relating to a patient at an instant of time and preferably on a live basis and also to make that data available for further analysis in isolation for a particular patient and/or in combination with data relating to a group of patients.

In a first aspect of the invention, there is provided a system for the monitoring of a condition of a person, said system including the provision of a mat having a surface which contacts with a support means and an opposing surface located with respect to at least part of the person so that at least one parameter of the person is monitored by sensor means provided with the mat and said sensor means are connected with processing means in order to allow data relating to the said at least one parameter to be collected and processed to generate a value which is indicative of the said parameter at the time of the data collection and wherein said mat includes a plurality of spaced apart sensor means locations.

In one embodiment, the mat includes a plurality of sensors to allow data from each of the sensors to be processed and combined in order to generate the value of the said parameter.

In one embodiment, the mat includes a plurality of different types of sensors, said different types of sensors provided so as to allow the data from the same to be collated and processed to generate a value for a particular parameter or alternatively to provide a value for a range of parameters relating to the condition of the person.

In one embodiment, the processing means allows data from the sensors to be used to provide a real time value relating to one or more parameters of the person's condition.

In another embodiment the processing means allows data from the sensors to be stored and then used as historical data for further analysis relating to the same or other parameters of the person's condition.

In one embodiment the values for the parameters in real time and/or historically can be combined to provide an indication of the general condition of the person.

In one embodiment, the mat is provided of a form so as to allow a surface of the same to be contacted or impacted by a person as they sit or lie on a support means, such as a chair or bed, and in relation to which the mat is located.

In one embodiment the person is lying down in a position such the rear of their body is closest to or in contact with the said mat surface.

In one embodiment, the configuration of the sensors with respect to the said mat surface and/or within the mat is selected in relation to different zones of the mat.

In one embodiment the outline of the respective zones are shaped such as to match the particular parts of the person's anatomy which are mostly likely to be located at each said zone. This is done so as to allow effective monitoring to be achieved in terms of the data generated and/or to ensure that sufficient data is obtained for particular parameters which are particularly relevant to the part of the person's anatomy with respect to which the zone is to be located.

For example, a zone which is located on the mat where the person's head is to be located may have a different configuration of sensors and/or different types of sensors located at that particular zone in comparison to another zones of said mat at which for example, the person's feet are to be located.

In one embodiment, the different configurations are selected so as to obtain a greater number of data readings from one zone than would be provided from another zone, for the same parameter.

In one embodiment the monitoring of the one or more parameters of a patient when on a support means such as a bed or chair is achieved by the provision on the mat of at least one layer of sheet material, a first set of lines of conductive material and a second set of lines of conductive material and said first and second sets of lines are angularly offset and the sensing means locations are formed at the intersections between said lines of the first and second sets of conductive material.

Typically the lines in each set are substantially parallel and the angular offset is such that the first set of lines are substantially perpendicular to the set of lines of the second sheet material.

Typically the first and second sheets of material are overlaid so as to form a conductive grid of sensing means locations in the mat.

In one embodiment the first and second sheets of material are spaced part by an intermediate layer on which the conductive lines are formed.

Typically the sheets of material are formed of non-woven fabric.

In one embodiment the conductive lines in each set are joined together in groups and connected together in groups row by row from the first sheet material and column by column from the second sheet material by ribbon connectors to processing means to allow the data from the sensing means location to be converted in to a parameter value.

The parameter values which are generated may be displayed to the end user numerically and/or visually.

In one embodiment, the said mat is provided to be located in or under one or more intermediate layers, such as a mattress of a bed, and the person lies or sits on the intermediate layer and the sensing means are still capable of obtaining the relevant data from the person through the intermediate layer.

In one embodiment, the mat is provided as a separate entity for use in conjunction with a mattress of any particular type and including air mattresses, or, the mattress can be provided as an integral part of the mat.

In one embodiment, the parameters which are provided allow the detection of any or any combination of the person's movement, temperature, pressure, humidity, tissue, oxygenation, and/or respiration to be detected and thereafter processed in order to generate an indication of one or more conditions of the person.

In one embodiment, the data which is received from said sensors, can be used to provide an immediate real time and/or historical indication, such as, for example, via one or more display screens provided at the particular location of the person and mat. Typically, a display screen which may be in the form of a tablet device can be provided for use with each mat at each bed.

In another embodiment the data from the sensors is processed locally and/or at remote location to which the sensors are connected to provide a real time and/or historical indication at a display station of a number of persons such as, for example, patients in a ward.

In another embodiment, the data from the sensors can be stored and processed so as to provide an indication of the patient's condition over a period of time and/or may be stored in a memory means database such as, for example, on the Cloud so as to provide and allow analysis of a number of persons conditions.

In any of the embodiments the data collected and/or values generated from subsequent processing can be made available to other interested parties for use, subject to data use legislation, and in this embodiment the data itself may not be linked to any identifiable persons.

Thus, in one embodiment, the data which is received, can be made available on a larger scale and in combination with data from other persons, to allow relatively large scale assessment of the condition of a group of persons to be performed and/or other actions such as the analysis of the performance of staff to be performed.

In one embodiment the data relating to the condition of one or more persons can be used for forensic purposes if there is a need to identify a problem with regard to the person's condition and when that problem occurred.

In one embodiment, in order to ensure the integrity and security of the data relating to particular persons, a particular security code may be required to allow access to be gained to data, values and the condition indication for the same.

It is envisaged that the data which is received from the mat can be used and presented to monitoring personnel in a number of different ways and at different levels of detail, for example, in one arrangement.

In one embodiment the sensors are provided in rows and columns within the mat structure and the location of each sensor can be identifiable.

In addition or alternatively at least one set of sensors may be provided as a layer of a material, such as an ink, on the mat by a suitable method such as printing or laminating.

In one embodiment the apparatus includes sensing means which act as a means of detecting data to be used in providing a value indicative of humidity.

In one embodiment the apparatus includes sensing means which act as a means of detecting data to be used in providing a value indicative of respiration.

In one embodiment the apparatus includes sensing means which act as a means of detecting data to be used in providing a value for spontaneous patient movement.

In one embodiment infra red sensors can be provided in order to allow data for the Oxygen level parameter to be obtained.

Typically data relating to environmental condition in which the person is located can be obtained and utilised in the generation by the processing means of the parameter value.

In one embodiment the method allows the capture and processing of details relating to a patient's assessment such as in accordance with the known assessment models by Braden or Waterlow.

Furthermore, in one embodiment, data relating to other events such as, records of the patient being turned, offloading and other interventions is stored.

In one embodiment in addition to monitoring parameters representing a patient's condition, said collected data can be combined and processed to provide further parameter values, such as parameters relating to risk of pressure damage to a patient if certain parameter values and/or combinations are detected and/or parameters that measure interventions with respect to the assessed risk.

In one embodiment the new parameter values representing risk and interventions for individual patients can be presented in various formats including, for example “dashboard” style displays, timelines, evaluation reports.

In a further aspect of the invention there is provided a mat for use in the generation of data representative of the condition of at least one parameter of a patient with respect to which the mat is located when the patient is on a support means in the form of a bed or chair, said mat formed of one or more sheet material layers, and sensing means locations are formed at spaced locations thereon to allow the sensing of a change in condition of said at least one parameter and to generate data representative of the said at least one parameter.

In one embodiment a first set of substantially parallel lines of conductive material and a second set of substantially parallel lines of conductive material are formed on the one or more sheet material layers and said first and second sets of lines are angularly offset and the sensing means locations are formed by the intersection of said lines. In one embodiment the angular offset is 90 degrees.

In one embodiment the said conductive material is provided by woven threads of conductive material or printed conductive material.

In a further aspect of the invention there is provided a method for the monitoring of a condition of a person, said method comprising the steps of providing a mat having a surface located with a support means and an opposing surface located so as to be impacted upon by at least part of the person when the person is on the support means, providing a number of sensing means locations on said mat operable to collect data relating to at least one parameter of the person, collecting and passing the said data to processing means and generating a value which is indicative of the said parameter at an instant of time.

In one embodiment the method includes the further steps of generating an indication of the condition of the said person based on said parameter values.

In one embodiment the said parameter values are used in real time and/or at a later time to generate the indication of the condition in respect of one and/or a number of persons.

In one embodiment the parameter values are used to generate a pressure map representative of the person.

In one embodiment the method is employed continuously whilst the person is in location with the support means with which the said mat is located.

In one embodiment the particular method and indications provided can be selected from different categories dependent upon the particular level of display and detail which is required.

Typically the method will include the use of an interface by the carer which allows the carer to identify the person's identity, obtain historical indications of their condition and/or real time indications.

In one embodiment the method includes during the processing of the data identifying the location or zone from which the data has been obtained and then, in one embodiment allocating different weightings or degrees of relevance to the data.

In one embodiment the same data may be used in developing indications for different parameters and in one embodiment different weighting values may be allocated for different parameter usage.

In one embodiment the processing of the data will make reference to other data such as anthropometric data relevant to the particular person.

Specific embodiments of the invention are now described with reference to the accompanying drawings; wherein

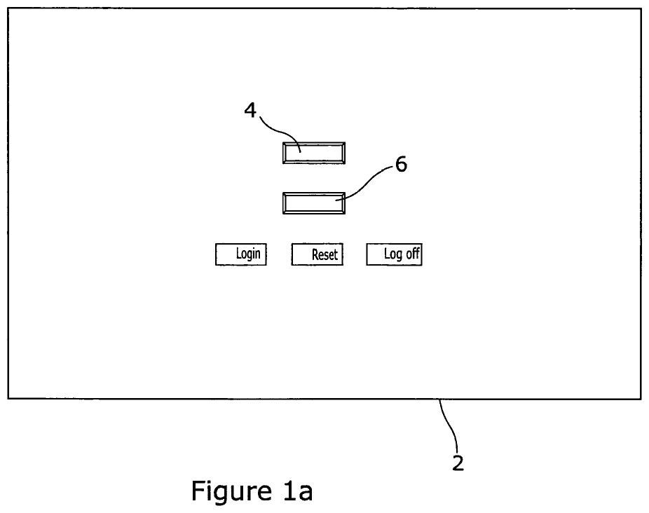

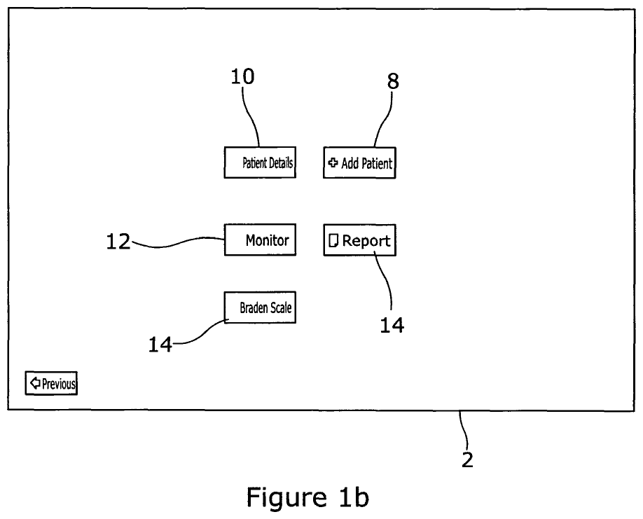

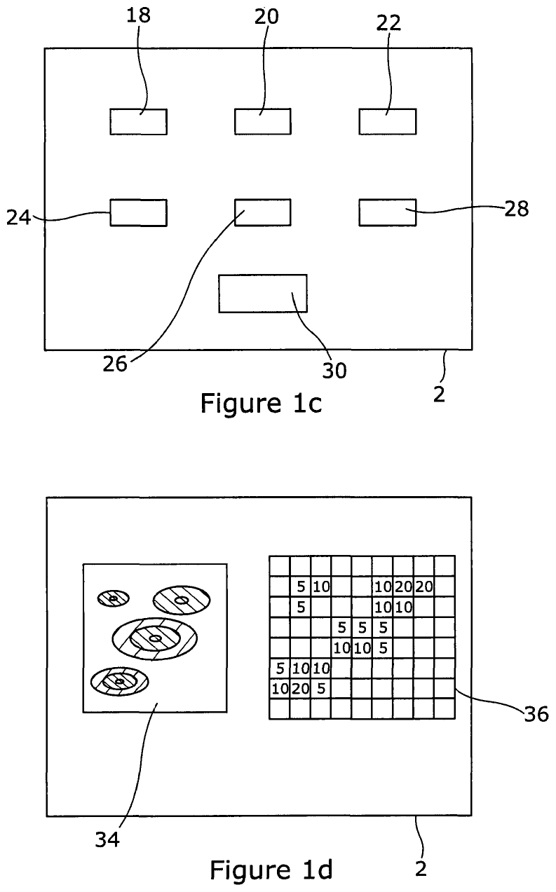

FIGS. 1a-d illustrate examples of user interface displays in accordance with one embodiment of the invention;

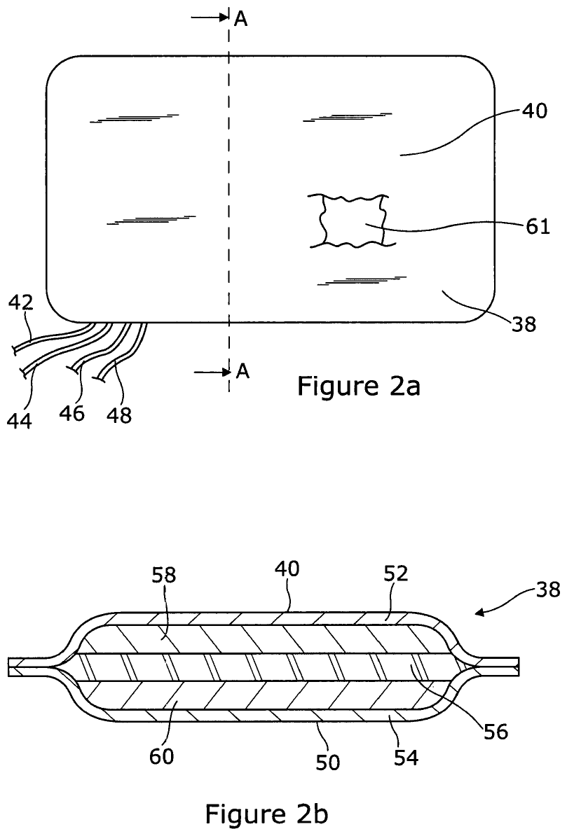

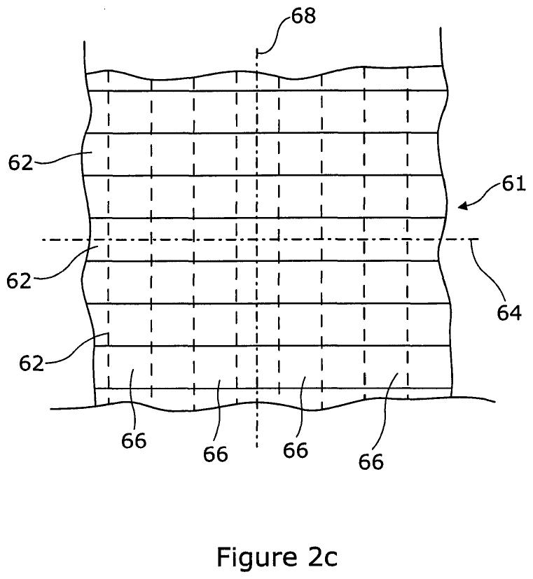

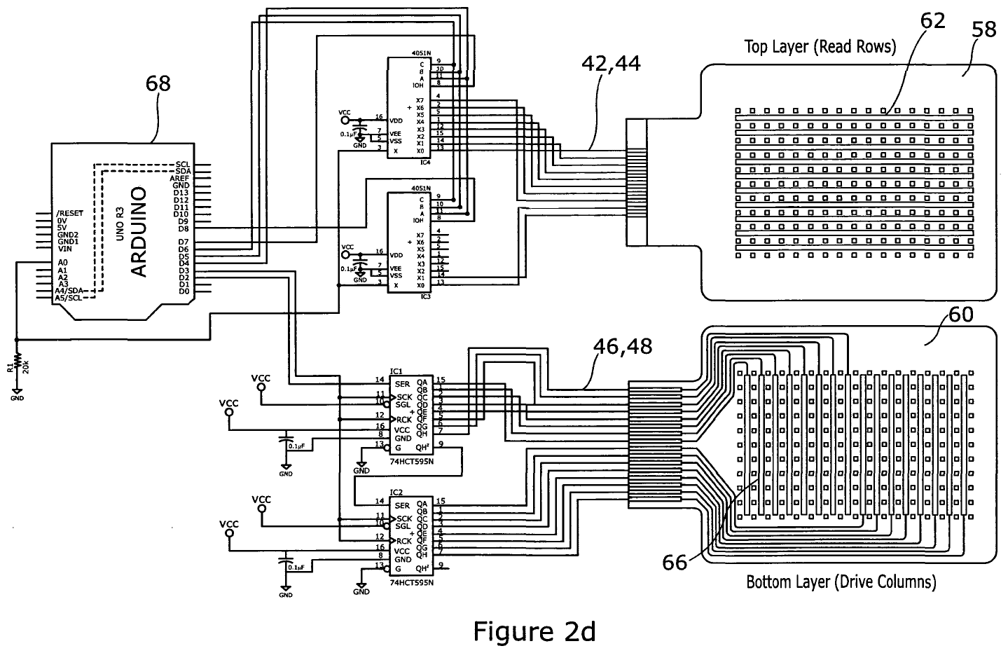

FIGS. 2a-d illustrate an example of the construction of a sensing means mat in accordance with one embodiment of the invention;

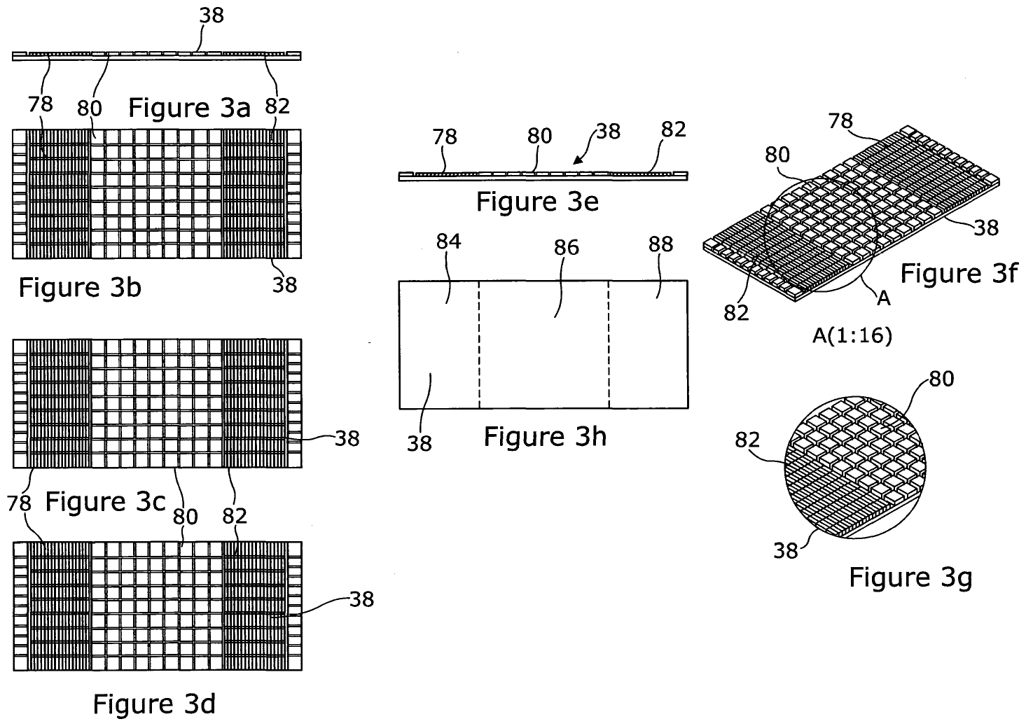

FIGS. 3a-h illustrate a mattress and mat combination in one embodiment of the invention;

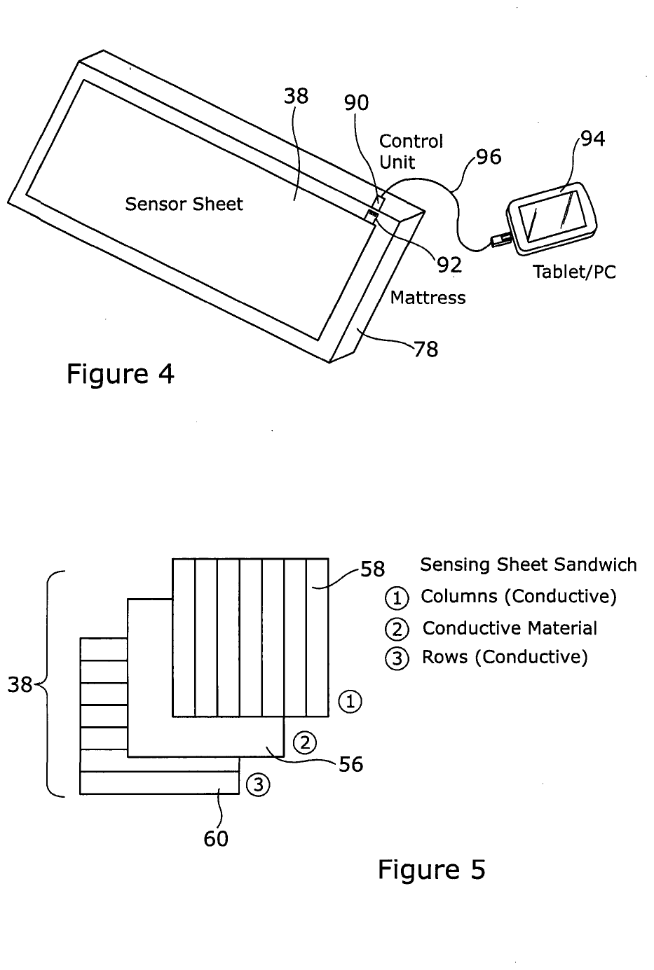

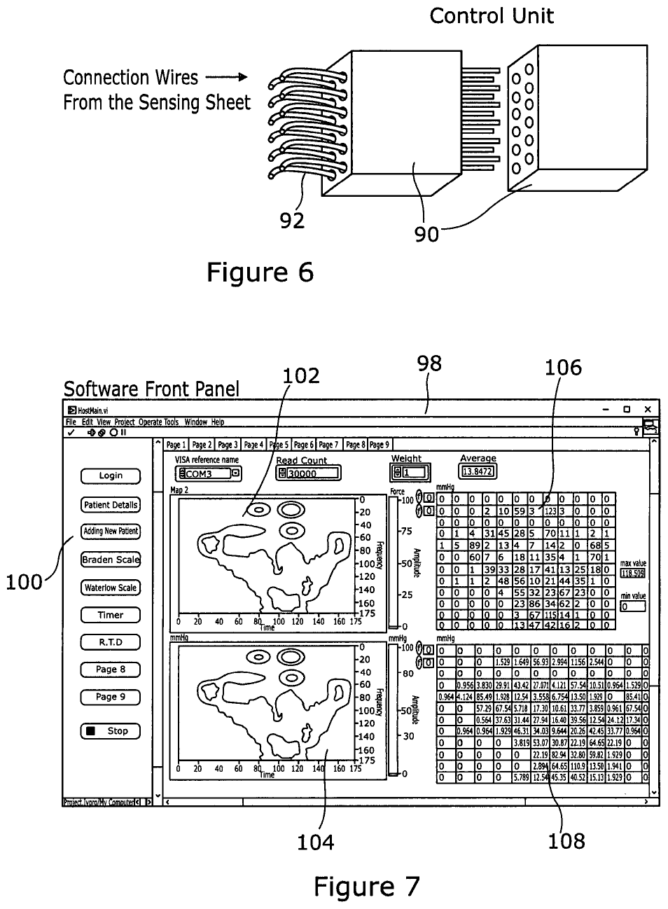

FIGS. 4-6 illustrate schematically components of the apparatus in accordance with one embodiment of the invention; and

FIG. 7 illustrates a further embodiment of a display for use in accordance with one embodiment of the invention.

Referring firstly to FIGS. 1a-d there is illustrated an example of a graphical user interface 2 provided on a display screen such as that of a lap top or PC. The GUI will have multiple display windows, four of which are indicated in the FIGS. 1a-d. Typically the GUI will be secured by a username and a password to be determined by the admin in the host company and this will be required to be input on the screen locations 4,6 as shown in FIG. 1a. Once access has been gained, the display shown in FIG. 1b is generated and this provides a number of options which, in this embodiment, include the ability to add a new patient's details 8, access or change existing patient details 10, access live and historical data relating to parameter and monitoring values for a patient or group of patients 12, generate a report 14 or to input data relating to a patient's pressure susceptibility condition 14 using a known scale which, in this example, is the Braden scale.

If the Braden scale option 14 is chosen then the display 16 shown in FIG. 1c is generated which allows the user to input a series of values in the locations 18-28 and on the basis of the input data a Braden scale value is calculated and display in location 30.

If the monitoring location 12 is selected then a display 32 is generated as shown in FIG. 1d. The format of the display may vary depending on the status of the user, so that for example, the display which is shown is that which would be generated for an administrator and shows both a visual display 34 showing regions and by different colours, pressure locations of the patient on a sensing mat and a numerical display 36 of the same parameter but which provides numerical values for the pressure locations and level of pressure applied. These displays can be selected to provide live information and/or historical information of the parameter.

Typically, at the same time the system will include an alarm which operates independently and at an agreed time period, such as every two hours, will display a message to alert the user of a need for action to be taken. Typically the alarm will continue until the required action is performed by the user.

One embodiment of sensing means which is used is provided in the form of a mat which is illustrated in FIGS. 2a-d. The mat 38 has a top surface 40 onto which a patient can directly or indirectly apply pressure at locations thereon, and an opposing surface which will contact with the mattress of a bed or a chair when in use. Connecting cables 42,44,46,48 depend therefrom to allow the transfer of data therealong which is indicative of the pressure applied and where it is applied.

The mat is formed, in one embodiment, by a plurality of layers shown in FIG. 2a which is a cross sectional side view along line AA. The outer layers 52, 54 are protective layers and form the outer surfaces 40, 50 of the mat. There is provided an intermediate layer 56 which is formed of a conductive fabric and which physically spaces first and second sheets of material 58, 60 both of which are provided with sets of parallel conductive lines formed therein. The conductive lines overlap at spaced locations to form sensing means locations and the pressure which is applied by the person when lying on the mat causes electrically conductivity values to be created at some or all of the sensing means locations, and each of the values is detected and passed to the data processing means. The location of each of the sensing means locations on the mat is known and the data therefrom is allocated to that particular location so that a sensing grid is formed across and along the mat. The particular level of the conductivity value at each sensing means locations changes as the person moves on the mattress and as the conductivity value can be allocated to a particular pressure level being applied to cause the electrical interface connection between the line of the first set and the line of the second set, and the location on the mat of that sensing means location is known, so a live pressure grid is created. As the greater the conductivity level which is achieved at a sensing means location indicates that a greater pressure is being applied at that location by the person then if a prolonged period of greater pressure is detected as being applied at the same sensing means locations this indicates a risk of pressure sores and an alarm can be generated to cause the carer to move the patient.

The number and width of the conductive lines can be selected to define the number of sensing means locations and hence the granularity and accuracy of detection which can be achieved by the mat but there may for example, by 48 lines in one set and 40 lines in the other set. In one example there are provided 2304 sensing means locations on the mat.

As illustrated in FIG. 2c, which is a view from the top surface 40 but with the outer layer 52 removed and which shows a portion 61 of the mat, the conductive lines 62 in the set of the first sheet material 58 are parallel with regard to the axis 64. The second sheet material 60 is arranged such that the conductive lines 66 in that set lie parallel to the axis 68 and substantially perpendicular to the conductive lines of the first sheet material 58 so as to form a sensing grid across the mat. Thus each grid square has an identifiable location and that can be matched with the electrical signal generated when pressure is applied at a particular location by the patient on the mat so as to generate an electrical signal via the conductive lines. The set of conductive lines 62 of the first sheet material 58 are schematically illustrated in the diagram of FIG. 2d and the set of conductive lines 66 of the second sheet material are also shown and which are shown as separate rather than overlaid in the diagram to illustrate the pin connections. The data acquisition is therefore achieved via cable connections 42,44 to a multiplexer for the rows of conductive lines 62 and via cable connections 46,48 to shift registers for the columns of conductive lines 66. In this way no information for the critical data of the patient is lost.

Typically some pins of the multiplexer will be grounded so as to avoid problems while reading the data and in one embodiment both the shift registers and multiplexers will be connected to pass the detected data from the mat to the processing platform 68 which may be in one embodiment an FPGA (Field Programmable Gate Array). Temperature and humidity sensors can also be incorporated into the circuitry shown and data from the same processed also to allow parameter values to be obtained.

In one embodiment of the apparatus the mat 38 provides is provided with different sensing zones so at to allow the ability to monitor interface pressures, temperature and humidity at 3 different zones of a mattress 76 with which the mat is to be used and FIG. 3 a-g illustrate such mattress 76 which has a head zone 78, a torso zone 80 and a leg and foot zone 82. In one embodiment the provision of different sensing zones 84, 86, 88 on the mat 38 are provided, as shown in FIG. 3h to match the location of the zones 78, 80, 82 of the mattress and the sensing means locations and /or sensing means types used in the different zones 84, 86, 88 of the mat may vary.

The apparatus can be provided with mains power operation or battery operated and in either case there will typically be an alarm system should the power supply fail and/or the charge level of the battery supply fall below a particular predetermined level.

In one embodiment Graphene material may be used as a conductive material for the mat.

In one embodiment the mat includes three zones for temperature/humidity sensing, i.e. head zone, body zone and leg zone. In one embodiment each zone may be provided with different sensing means types, different sensing means locations configurations and/or different data processing requirements. In one embodiment connection of data may be achieved using a 8×4 multiplexer and/or wirelessly, via for example Bluetooth.

In an alternative embodiment the mat is formed of a single layer with conductive sensing means locations provided in a required configuration thereon.

In one embodiment the apparatus operates continuously by scanning the sensing means locations on the mat in a continuous and sequential manner along the mat length and/or across the width.

In one embodiment, in order to sense the temperature and/or humidity, a sensor, such as a DHT 22 sensor is located in each zone, and in one embodiment may be surface mounted on the mattress or chair.

In one embodiment sensing means and/or data processing capability is provided to allow the measurement of shear and friction i.e. movement of the person's body across the bed surface.

In one embodiment of use of the apparatus an embodiment of which is illustrated schematically in FIGS. 4-6, the cover of the mattress 78 of the bed with which the mat 38 is to be used is unzipped and the textile sensor mat 38 is placed above the mattress 78 so as to lie between the mattress and the mattress cover. In one embodiment the sheet material layer or layers of the mat is formed of a conductive central layer 56 and on either side layers 58, 60 with conductive thread located thereon so as to form the sets of conductive lines and at their points of intersection or overlying via the layer 56, the sensing means locations.

The control unit 90 for the mat remains available to use and is connected to the output connections 92 of the mat so as to connect to the sensing means locations of the mat. The control unit may include or is connected to the data processing means and a display device 94 by a cable connection 96 or could be wirelessly connected.

A software package to control the data processing, display options and control of the mat is provided and the package may be designed to suit particular price levels, use requirements and capabilities. There may also be provided different levels of accessibility due to concerns over the persons data security and confidentiality and hence security code access to the apparatus and the processed data will typically be required. Another embodiment of a front display panel 98 is shown in FIG. 7 which illustrates on the left hand column 100 a range of possible options which can be accessed by the carer, if authorised, display screens 102, 104 which illustrate graphically the live condition of the one or more parameters of the patient which are being monitored and in grids 106, 108 the sensing means values for the parameter for each sensing location on the mat. These displays can be selected to show data from different zones of mat or may show a live feed and an historical feed, so as to allow a comparison to be made and these can be selected to suit the carer's requirements.

Different access levels are possible in case of security and confidentiality concerns and for different usage requirements. For example, at one access level there is provided a Patient Details function which allows the carer to be directed to another window, where they can enter patient detail and save them within the record so that all of the data which is generated in this use will be allocated to a particular patients record. Typically, at the start of each use the carer can add a new patient record and save the new data within the patients details within the record and within the caring organisation.

On another level, a patient reference system can be accessed and used, such as the Braden and Waterlow scales help assess the pressure risk of the patient using this scale.

A timer function allows the carer to set a time limit after which , if no movement of the patient is detected in that time, will causes the generation of an indicator and a message to the carer to check the patient. The time can be adjustable depending on the patient status and it counts the time by minutes.

The apparatus and method allows real time data monitoring to be performed of, for example, the real-time pressure of the patient and can generate a map of the pressure and where it is the peak pressure, which allows the carer to understand the pain of the patient and try to relieve the pressure by checking the map of the patient.

A history track function allows the carer to keep the monitoring of the patient in real-time and choose to save the best map picture of the patient to compare it later with the oldest picture and check if there is any progression in the patients condition.

A real time report can be generated including selected details of the patient at a particular instant of time. There is therefore provided full traceability of the patient's time spent on the mattress from admission to discharge. A clinical assessment tool can be incorporated and accessed remotely online to allow the remote monitoring of clinical and product service needs.

In one embodiment the mat can be provided integrally with the support means such as a mattress/bed, chair, operating table, X-ray table or trolley in a medical care environment.

There is therefore provided in accordance with the invention a monitoring system to prevent and monitor several parameters relating to a person's, and in particular a patient's, condition and to allow risks, such as the occurrence of pressure ulcers on patients to be reduced, whilst, at the same time reducing the need for personnel and thus providing cost savings.

1. Apparatus for monitoring of a condition of a person, said apparatus comprising:
a provision of a mat having a surface which contacts with a support means and an opposing surface located with respect to at least part of the person so that at least one parameter of the person is monitored by a plurality of sensor means provided at locations with the mat and said plurality of sensor means are connected with processing means in order to allow data relating to the said at least one parameter to be collected and processed to generate a value which is indicative of the said parameter at a time of the data collection and wherein said mat includes a plurality of spaced apart sensor means locations.

2. Apparatus according to claim 1 wherein data from the plurality of sensor means locations is combined and processed in order to generate a value for the said at least one parameter.

3. Apparatus according to claim 1 wherein the apparatus includes a plurality of different types of sensor means to allow data for different parameters of the patient to be provided and processed to generate values for said different parameters and/or a value representative of the persons condition as defined by the plurality of said parameter values.

4. Apparatus according to claim 1 wherein the one or more values provide a real time indication of the at least one parameter of the person's condition.

5. Apparatus according to claim 1 wherein the mat is provided of a form to allow the plurality of spaced apart sensor means locations thereon to be contacted and/or impacted upon by the person when they sit or lie on the mat or on the support means in relation to which the mat is located.

6. Apparatus according to claim 1 wherein the mat is provided with two or more sensing zones which have different sensing capabilities.

7. Apparatus according to claim 6 wherein the said two or more sensing zones are located along an axis of the mat along which the person is substantially located when lying down on the mat or support means with which the mat is provided.

8. Apparatus according to claim 6 wherein a configuration of the spacing and/or number of the sensor means locations with respect to the said mat is selected with regard to the said two or more sensing zones of the mat.

9. Apparatus according to claim 6 wherein the two or more sensing zones are sized so as to match particular parts of the person's anatomy which are most likely to be located at each said two or more sensing zone when the person is located with the mat.

10. Apparatus according to claim 1 wherein the apparatus includes sensing means to allow pressure, temperature and/or humidity to be detected.

11. Apparatus according to claim 10 wherein at least the pressure sensing means are located at the said sensing means locations on the said mat.

12. Apparatus according to a claim 1 wherein the said mat is located between the support means for the person on one side and one or more layers on the opposing side and which are intermediate the mat and the person when sitting or lying on the support means.

13. Apparatus according to claim 12 wherein the support means is a bed or chair with a support surface for the person.

14. Apparatus according to claim 13 wherein the said mat is provided to be selectively placed on or into the support means or as an integral part of said support means.

15. (canceled)

16. Apparatus according to claim 1 wherein the parameters for which data is collected and/or processed include any, or any combination, of movement, temperature, pressure, humidity, tissue, oxygenation and/or respiration.

17. Apparatus according to claim 1 wherein the apparatus includes a display screen to allow data and/or subsequently processed data to be viewed via the said display screen which is located remotely from said mat or located as part of the mat and/or support means with which the mat is provided.

18. Apparatus according to claim 1 wherein the plurality of spaced apart sensing means locations on the mat are provided in a grid configuration.

19. Apparatus according to claim 1 wherein the mat includes a first set of lines of conductive material and at least a second set of lines of conductive material, said second set angularly offset to the first set of lines and wherein where the points of intersection of said lines define the said plurality of spaced apart sensing means locations on the mat and connectors connect the said lines of conductive material and hence the sensing means locations to allow the transfer of sensing data to the processing means.

20. (canceled)

21. (canceled)

22. Apparatus according to claim 19 wherein the mat is formed of a fabric and lines of conductive material thereon are woven threads or printed conductive material.

23. A mat for use in the generation of data, representative of a condition of at least one parameter of a patient with respect to which the mat is located when the patient is on a support means in the form of a bed or chair, said mat formed of one or more sheet material layers, and sensing means locations are formed at spaced locations thereon to allow the sensing of a change in condition of said at least one parameter and to generate data representative of the said at least one parameter said mat including a first set of substantially parallel lines of conductive material and a second set of substantially parallel lines of conductive material are formed on the one or more sheet material layers and said first and second sets of lines are angularly offset and the sensing means locations are form by the intersection of said lines.

24-26. (canceled)

27. A method for the monitoring of a condition of a person, said method comprising the steps of:
providing a mat having a surface located with a support means and an opposing surface located so as to be impacted upon by at least part of a person when the person is on or located on one or more layers which are located on the support means;
providing a number of sensing means locations on said mat which are operable to collect data relating to at least one parameter of the person;
collecting and passing the said data to processing means; and
generating a value which is indicative of the said parameter at an instant of time.

28. A method according to claim 27 wherein a plurality of different types of sensing means are provided to allow the collection of data relating to a plurality of parameters relating to the condition of said person.

29. A method according to claim 27 wherein the processing means allows data from the sensors to be stored and then used as historical data for further analysis relating to the same or other parameters of the person's and/or a group of said persons conditions.

30. A method according to claim 29 wherein the values for the one or more parameters in real time and/or historically, are combined to provide an indication of the general condition and/or care of the person over a period of time.

31. A method according to claim 27 wherein the parameter values are used to generate a pressure map representative of at least the part of the person located with respect to said mat.

32. (canceled)
